(19) United States
(12) Patent Application Publication
(10) Pub. No.: US 2017/0135590 A1
(43) Pub. Date: May 18, 2017

(54) MAGNETIC RESONANCE IMAGING APPARATUS AND BLOOD FLOW DRAWING METHOD

(71) Applicant: HITACHI, LTD., Tokyo (JP)

(72) Inventor: Hiroyuki ITAGAKI, Tokyo (JP)

(73) Assignee: HITACHI, LTD., Tokyo (JP)

(21) Appl. No.: 15/319,534

(22) PCT Filed: Jul. 2, 2015
(86) PCT No.: PCT/JP2015/069110

§ 371 Date: Dec. 16, 2016

(30) Foreign Application Priority Data
Jul. 15, 2014 (JP) 2014-145358

(51) Int. Cl. A61B 5/026; G01R 33/567; G01R 33/483; G01R 33/563; A61B 5/00; A61B 5/0456

(52) U.S. Cl. CPC A61B 5/0263; A61B 5/7475; A61B 5/0037; A61B 5/742; A61B 5/0456; G01R 33/4833; G01R 33/56316; G01R 33/5673

(57) ABSTRACT

To obtain an image having high ability of imaging a blood vessel in each cardiac time phase when performing imaging through a cine-PC method, an MRI apparatus includes a magnetic resonance imaging unit that collects a magnetic resonance signal; a control unit that controls the magnetic resonance imaging unit as per a pulse sequence; and a signal processing unit that prepares an image of a test target by using the magnetic resonance signal collected by the magnetic resonance imaging unit, and time phase information related to a motion of the test target. The control unit has an imaging sequence which serves as the pulse sequence, which includes applying of a flow encoding pulse, and in which an echo signal is acquired for each time phase. An applying amount of the flow encoding pulse in the imaging sequence is controlled so as to be different depending on the time phase.

### TECHNICAL FIELD

The present invention relates to a vascular imaging technology based on a phase contrast angiography method (hereinafter, PC method) in a magnetic resonance imaging (hereinafter, will be referred to as MRI) apparatus in which a nuclear magnetic resonance (hereinafter, will be referred to as NMR) signal from hydrogen, phosphor, or the like in an object is measured, and density distribution of nuclei, distribution of relaxation times, and the like are image-formed, and particularly relates to a cine-PC method in which imaging is continuously performed in a time series.

### BACKGROUND ART

In MR angiography which is a technology of imaging a blood vessel using an MRI apparatus, there is a PC method in which a blood flow is image-formed by using the principle of a phase of transverse magnetization of blood shifting in accordance with a blood flow velocity (PTL 1). In the PC method, since a phase shift is applied to a spin having a velocity, a gradient magnetic field which has bipolarity and is called a flow encoding pulse is used. A complex differential between an image acquired by applying a flow encoding pulse having the positive polarity and an image acquired by applying a flow encoding pulse having the negative polarity is taken, and a vascular image having a value of the flow velocity reflected is obtained.

The phase shift generated in a spin depends on an applying amount (flow encoding amount) of the flow encoding pulse and the velocity of a blood flow. Therefore, when an appropriate flow encoding amount is set with respect to a blood flow which is a target of imaging, the blood flow can be imaged with high luminance. In addition, since the amount of the phase shift depends on the blood flow velocity, the blood flow velocity can be obtained from a phase image acquired through the PC method by utilizing the dependence thereof.

As described above, in the PC method, an appropriate flow encoding amount is required to be set in accordance with the blood flow velocity of a target blood vessel. Generally, in the MRI apparatus, when the PC method is executed, a user sets a value (called VENC) corresponding to a desired blood flow velocity, thereby setting the flow encoding amount. In order to image all of multiple blood vessels having different blood flow velocity with high luminance, the technology in PTL 1 discloses a technique of a composite image prepared for each VENC by setting multiple VENCs and using an echo signal measured at each VENC.

The PC method is suitable for imaging the blood flow velocity, thereby being also applied to cine-imaging in which a vascular image is acquired at different timing within a cardiac cycle and a change of a blood flow within the cardiac cycle is imaged (PTL 2). In the cine-imaging (hereinafter, will be referred to as cine-PC imaging) performed through the PC method, for example, the blood flow velocity related to the cardiac cycle such as an early stage and a late stage in a systolic stage, an early stage and a late stage in a diastolic stage, and the like can be imaged. Therefore, in the technology disclosed in PTL 2, information of the blood flow velocity of a cardiac time phase obtained in the cine-PC imaging is utilized in vascular imaging in an image obtained through a different imaging sequence.

### CITATION LIST

### Patent Literature

PTL 1: Japanese Patent No. 5394374
PTL 2: Pamphlet of International Publication No. 2011/132593

### Non Patent Literature

NPL 1: Proc. Intl. SOc. Mag. Reson. Med. 20 (2012) “Selective TOF MRA using Beam Saturation Pulse

### SUMMARY OF INVENTION

### Technical Problem

As described above, in a PC method, a flow encoding amount is set in accordance with a blood flow velocity of a blood vessel as an imaging target or a mean blood flow velocity of multiple blood vessels flowing in target tissue. However, in a case where cine-imaging of a blood vessel of the heart or in the vicinity thereof is performed, the blood flow velocity flowing therein significantly varies in response to a cardiac cycle.

Therefore, for example, in a case of using one flow encoding amount with reference to the mean flow velocity of the cardiac cycle or the maximum flow velocity, for example, a target blood vessel is imaged with high luminance in an early stage of contraction. However, target blood vessel may be imaged with low luminance in periods other than thereof. Therefore, in a case where the blood flow velocity obtained through cine-PC imaging is analyzed and the measurements of vascular movement and the like are calculated, the measurements including the blood flow velocity cannot be accurately obtained.

PTL 1 discloses a technology of performing imaging with multiple VENC values in consideration of the blood flow velocity of multiple blood vessels having different blood flow velocity. However, the technology cannot cope with a problem of deterioration of the ability of imaging a blood flow in the cine-imaging having a temporally changing blood flow as a target.

The present invention aims to obtain an image having high ability of imaging a blood vessel in each cardiac time phase when performing imaging through a cine-PC method. In addition, the present invention also aims to obtain a cine-image which has high ability of imaging a blood vessel and in which a temporal change of the blood flow velocity can be grasped.

### Solution to Problem

In order to solve the problems described above, according to the present invention, there is provided an MRI apparatus which is provided with a function of changing setting of a VENC value for each cardiac time phase in imaging performed through a cine-PC method. In other words, the MRI apparatus of the present invention includes a magnetic resonance imaging unit; a control unit that controls the magnetic resonance imaging unit as per a pulse sequence; and a signal processing unit that prepares an image of a test target by using a magnetic resonance signal collected by the magnetic resonance imaging unit, and time phase information related to a cyclic motion of the test target. The control unit has an imaging sequence which serves as the pulse sequence, which includes applying of a flow encoding pulse, in which an echo signal is acquired for each time phase. An applying amount (flow encoding amount) of the flow encoding pulse in the imaging sequence is controlled so as to be different in at least two time phases.

In addition, According to the present invention, there is provided a blood flow imaging method in which a magnetic resonance image for each time phase is acquired by executing a pulse sequence including a flow encoding pulse with reference to time phase information related to a cyclic motion of a test target. In the blood flow imaging method, an applying amount of the flow encoding pulse is caused to be different in at least two time phases. The applying amount of the flow encoding pulse is caused to be different in accordance with a blood flow velocity of a blood flow flowing in the test target.

### Advantageous Effects of Invention

According to the present invention, in cine-PC imaging, a flow encoding amount of each cardiac time phase is optimized, and the ability of imaging a blood vessel and the measurement accuracy of the blood flow velocity are improved.

### BRIEF DESCRIPTION OF DRAWINGS

FIG. 1 is a view illustrating an overall configuration of an MRI apparatus to which the present invention is applied.

FIG. 2 is a functional block diagram of a control unit and a computation unit.

FIG. 3 is a view illustrating an example of a pulse sequence of a PC method.

FIG. 4 is a view illustrating a cine-PC sequence using the pulse sequence of the PC method in FIG. 3.

FIG. 5 is a view illustrating a change of a blood flow velocity in one cardiac cycle.

FIG. 6 is a flow illustrating operations of the control unit and the computation unit of a first embodiment.

FIG. 7 is a view illustrating a pre-scanning sequence used in the first embodiment.

FIG. 8 is a flow illustrating details of processing included in the flow of FIG. 6.

FIGS. 9(a) to 9(c) are views respectively illustrating pieces of pre-scanning data being processed.

FIGS. 10(a) and 10(b) are views respectively illustrating relationships between a time phase in main imaging and a time phase in pre-scanning of a second embodiment.

FIG. 11 is a view illustrating a sequence of a two-dimensional space selection excitation method used as the pre-scanning of a third embodiment.

FIG. 12 is a flow illustrating operations of the control unit and the computation unit of the third embodiment.

FIG. 13 is a view illustrating a UI for designating a two-dimensional excitation region in the pre-scanning of the third embodiment.

FIG. 14 is a view illustrating a relationship between the time phase in the main imaging and the time phase in the pre-scanning of the third embodiment.

FIG. 15 is a view describing a retrospective imaging method employed in a fourth embodiment.

FIG. 16 is a view illustrating an execution form of a GUI which is used in common in the embodiments.

### DESCRIPTION OF EMBODIMENTS

An MRI apparatus of the present embodiment includes a magnetic resonance imaging unit that collects a magnetic resonance signal; a control unit that controls the magnetic resonance imaging unit as per a pulse sequence; and a signal processing unit that prepares an image of a test target by using the magnetic resonance signal collected by the magnetic resonance imaging unit, and time phase information related to a cyclic motion of the test target. The control unit has an imaging sequence (cine-PC sequence) which serves as the pulse sequence, which includes applying of a flow encoding pulse, and in which an echo signal is acquired for each time phase. An applying amount of the flow encoding pulse in the imaging sequence is controlled so as to be different depending on the time phases.

In addition, in the MRI apparatus of the present embodiment, the signal processing unit includes a pulse calculating section which calculates the applying amount of the flow encoding pulse for each time phase based on velocity information of a fluid included in the test target. The control unit executes the imaging sequence including the flow encoding pulse with reference to the applying amount of the flow encoding pulse calculated by the pulse calculating section.

Hereinafter, with reference to the drawings, the MRI apparatus of the present embodiment will be described.

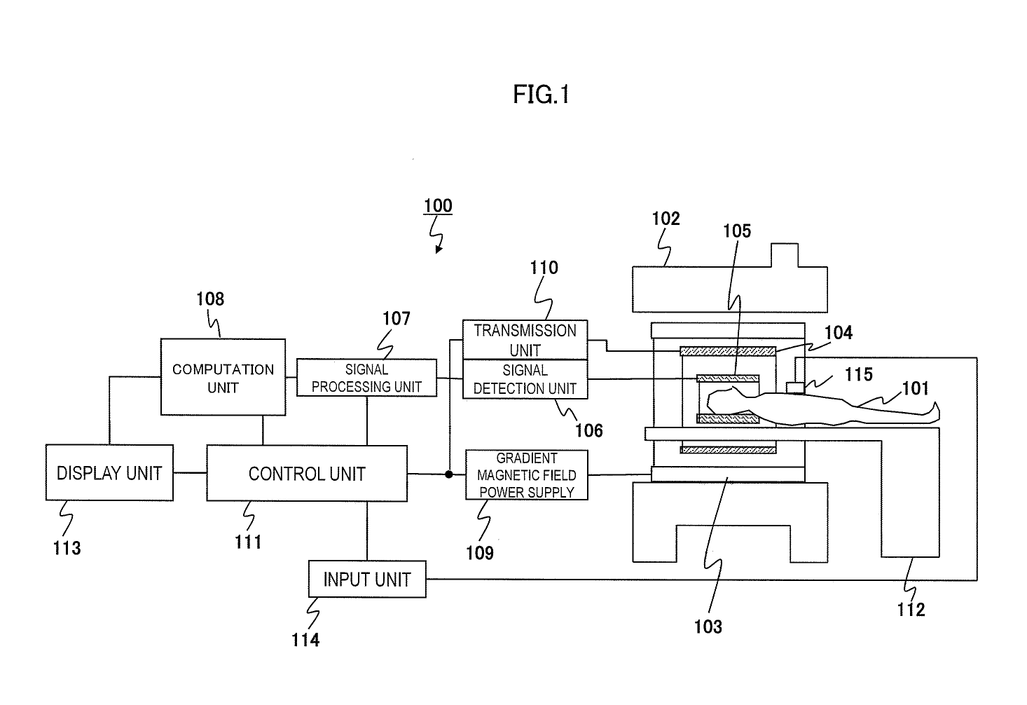

FIG. 1 is a configuration diagram of the MRI apparatus of the present embodiment. As illustrated in FIG. 1, according to the present embodiment, an MRI apparatus 100 serving as the magnetic resonance imaging unit includes a bed 112 on which an object 101 lies down, a magnet 102 which generates a static magnetic field in a space where the object 101 is placed, a gradient magnetic field coil 103 which generates a gradient magnetic field in the space where the static magnetic field is generated, a gradient magnetic field power supply 109 which supplies electricity to the gradient magnetic field coil 103, an RF coil 104 which applies a high frequency magnetic field to the object 101, a transmission unit 110 which supplies a high frequency signal to the RF coil 104, an RF probe 105 which receives a nuclear magnetic resonance signal (MR signal) generated by the object 101, a signal detection unit 106 which detects the signal received by the RF probe 105, and a signal processing unit 107 which performs predetermined signal processing with respect to the MR signal.

The MRI apparatus 100 further includes a computation unit 108 which performs computation of image reconstruction and the like by using a signal received from the signal processing unit 107; a control unit 111 which controls operations of the signal detection unit 106, the signal processing unit 107, the transmission unit 110, and the like; a display unit 113 which displays an image and the like; and an input unit 114 for inputting a command or information required in controlling of the control unit 111. The RF coil 104 and the RF probe 105 are disposed in the vicinity of the object 101. In FIG. 1, the RF coil 104 and the RF probe 105 are illustrated as separate devices. However, one coil may serve as an RF transmitting and receiving coil.

The gradient magnetic field coil 103 is configured with a gradient magnetic field coil of three directions such as X, Y, and Z. The gradient magnetic field coil 103 generates a gradient magnetic field of directions of three axes orthogonal to each other, in accordance with a signal from the gradient magnetic field power supply 109. The transmission unit 110 includes a high frequency oscillator and an RF amplifier and sends a signal to the RF coil 104 based on controlling of the control unit 111. Accordingly, a high frequency magnetic field pulse having a predetermined pulse shape is applied from the RF coil 104 to the object 101. A high frequency magnetic field generated from the object 101 in response to the high frequency magnetic field pulse is received by the RF probe 105 as an echo signal. The signal detection unit 106 and the signal processing unit 107 include an orthogonal detection circuit, an A/D converter, and the like. The signal detection unit 106 and the signal processing unit 107 detect the echo signal received by the RF probe 105 and impart the echo signal to the computation unit 108, as MR signal data which is a digital signal.

The computation unit 108 performs processing such as correction processing and Fourier transformation with respect to the MR signal data and generates display data such as an image and a spectrum waveform. In the present embodiment, the computation unit 108 has a function of calculating conditions required in imaging, in addition to a function of generating the display data described above.

The display unit 113 displays an image and the like prepared by the computation unit 108. The input unit 114 includes an input device such as a keyboard and a mouse, thereby receiving an input of a command from an operator. In addition, the input unit 114 inputs information from a measurement instrument 115 attached to the object 101 and imparts the information to the control unit 111. Examples of the measurement instrument 115 include a body motion meter which measures body motion, a pulse wave meter which measures cardiac motion, and an electrocardiograph, which are suitably mounted on the object 101 in accordance with the purpose of imaging. In the present embodiment, the measurement instrument 115 measuring a cardiac cycle is employed, and information (time phase information) from the measurement instrument 115 is taken into the control unit 111 via the input unit 114. The display unit 113 and the input unit 114 also serve as interfaces for inputting a command from an operator, for example, setting of object information and imaging conditions, and executing and stopping the imaging.

The control unit 111 converts the input imaging conditions into a timing chart related to applying of a magnetic field. As per the timing chart, the control unit 111 controls the gradient magnetic field power supply 109, the transmission unit 110, and the signal detection unit 106, thereby executing imaging. The time chart of controlling is called a pulse sequence. The pulse sequence has various items programmed in advance in accordance with the purpose of imaging. The pulse sequence is stored in a memory provided in the control unit 111. In the present embodiment, a pulse sequence of a PC method is used as the pulse sequence.

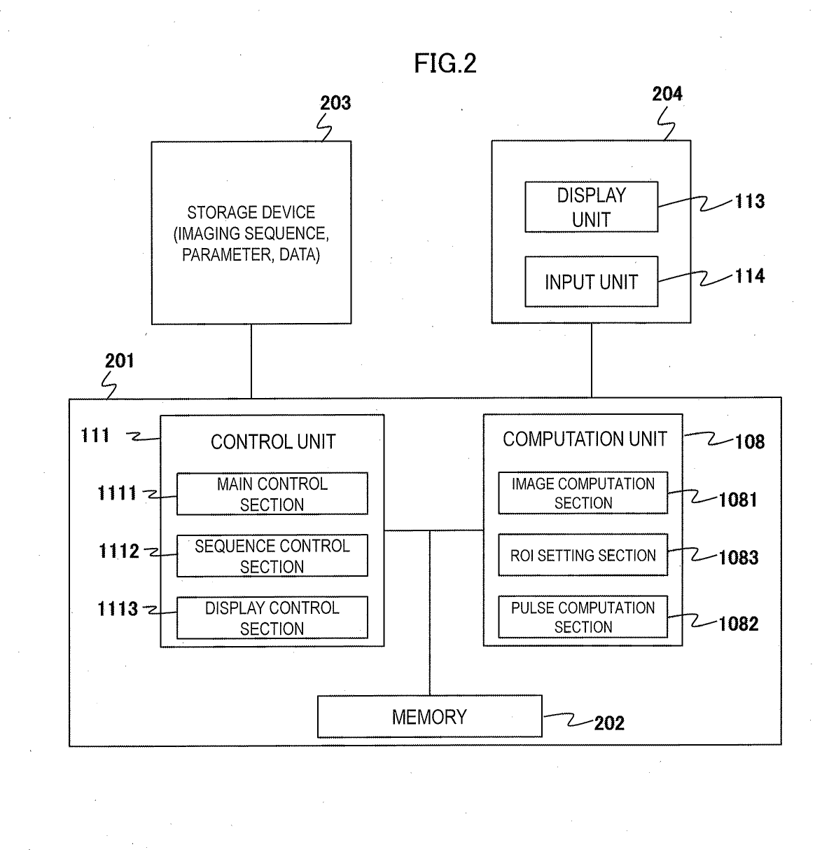

FIG. 2 is a block diagram illustrating functions of the control unit 111 and the computation unit 108. As illustrated, the control unit 111 includes a main control section 1111 which controls the operation of the apparatus in its entirety, a sequence control section 1112 for executing imaging as per the pulse sequence, and a display control section 1113 which controls displaying of the display unit 113. The computation unit 108 includes an image computation section 1081, a pulse computation section 1082, and an ROI setting section 1083 which sets a region to be a target of computation. The pulse computation section 1082 performs calculating the applying amount of a pulse, particularly, the applying amount of the flow encoding pulse, and normalization processing and the like with respect to data for each time phase in cine-imaging (functions as a normalization coefficient calculating section).

Each unit of the control unit 111 and the computation unit 108 can be established in a system including a CPU 201, a memory 202, a storage device 203, and a user interface 204. The function of each unit can be realized when a program stored in the storage device 203 in advance is loaded to the memory 202 and is executed by the CPU 201. In addition, a part of the function can be configured with hardware such as an application specific integrated circuit (ASIC) and a field programmable gate array (FPGA).

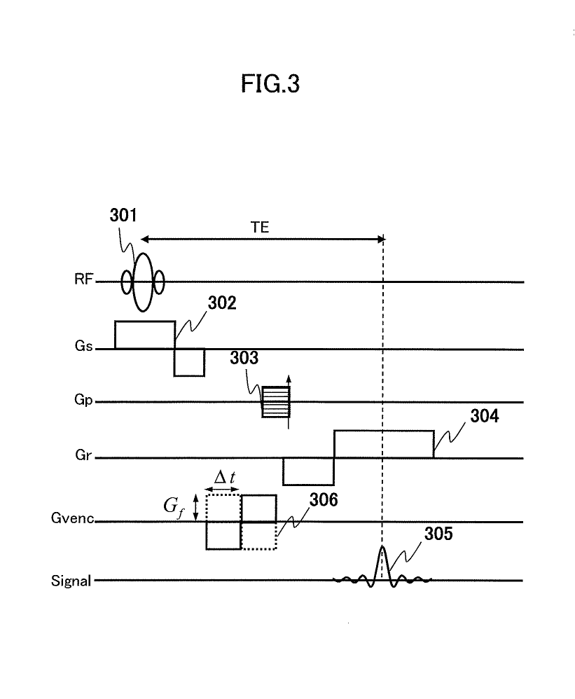

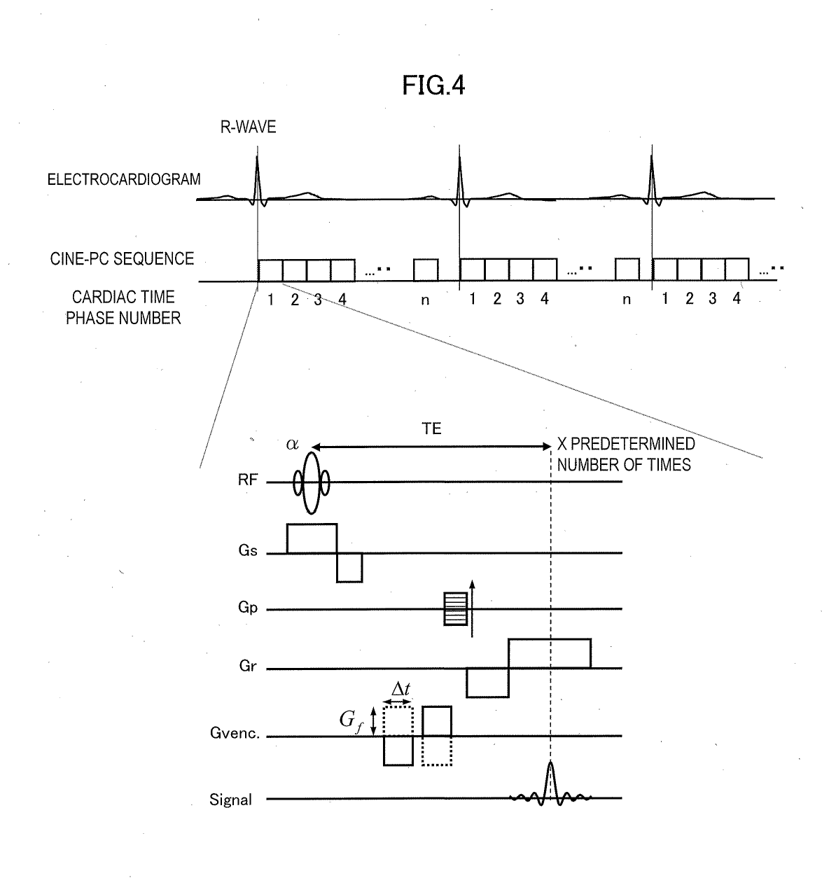

Subsequently, cine-imaging using the pulse sequence of the PC method employed in the MRI apparatus of the present embodiment will be described with reference to FIGS. 3 and 4.

FIG. 3 is a view, as an example of the pulse sequence of the PC method, illustrating a pulse sequence of a two-dimensional gradient echo (GrE) method as much as one repetitive time (TR). FIG. 4 is a time chart describing the cine-imaging. In FIG. 3, RF, Gs, Gp, Gr, Gvenc, Signal respectively indicate axes of an RF pulse, a slice gradient magnetic field, a phase encoding gradient magnetic field, a frequency encoding gradient magnetic field, a flow encoding gradient magnetic field, and an echo signal.

In the pulse sequence of FIG. 3, an RF pulse 301 is applied as well as applying of a slice gradient magnetic field 302, and a desired region of an object is selectively excited. Succeedingly, a phase encoding gradient magnetic field 303 is applied, and a frequency encoding gradient magnetic field 304 having an inverted polarity is applied. At the time point when the applying amounts of the frequency encoding gradient magnetic field 304 having the negative polarity and the frequency encoding gradient magnetic field 304 having the positive polarity become the same as each other, an echo signal 305 forming the peak is measured within a predetermined sampling time. The above-described process from applying the RF pulse 301 to measuring the echo signal 305 is the same as that in the pulse sequence of a basic GrE method. However, in the pulse sequence of the PC method, a flow encoding pulse 306 is added thereto.

The flow encoding pulse 306 has an effect of causing the phase of the fluid present within an excitation region, mainly a blood flow spin to be different from that of the spin of a stationary portion. As the axis Gvenc thereof, one to three desired axes in an X-direction, a Y-direction, and a Z-direction are selected in accordance with the flowing direction of the fluid.

The flow encoding pulse 306 includes a pulse (will be referred to as the flow encoding pulse having the positive polarity) indicated with the solid line in FIG. 3, and a pulse (will be referred to as the flow encoding pulse having the negative polarity) indicated with the dotted line. Each thereof includes a pair of positive and negative gradient magnetic fields. In the pair of positive and negative gradient magnetic fields, only the polarities are different from each other, and the applying amounts (absolute values) are equal to each other. In addition, the applying amounts of the flow encoding pulse having the positive polarity and the flow encoding pulse having the negative polarity are equal to each other. An applying amount S of a pulse is the product of a strength Gf and an applying time Δt when the strength Gf of a pulse is uniform. Blood vessel imaging is performed by repeating the echo signal measurement in which only the flow encoding pulse having the positive polarity is used, and the echo signal measurement in which only the flow encoding pulse having the negative polarity is used.

In repeating the pulse sequence (one repeating unit) of FIG. 3, for example, measurement using the flow encoding pulse having the positive polarity and measurement using the flow encoding pulse having the negative polarity are consecutively performed with the same phase encoding. While having the measurements as one set and changing the phase encoding, the measurements of one set are repeated until the echo signals of all of the set phase encoding are measured.

The flow encoding pulse included in the pulse sequence of the PC method described above is a pulse which applies a phase change to transverse magnetization. When the applying amount (flow encoding amount) thereof is set to an appropriate value, the difference between the phase of the spin of a blood flow in a direction parallel to the axis thereof and the phase of the spin of the stationary portion can be increased, and thus, the ability of imaging a blood flow can be enhanced. When the velocity of a blood flow is V, a phase shift amount φf the blood flow spin flowing in the direction parallel to the axis of the flow encoding pulse is expressed through the following Expressions (1) and (2). Expression (1) is a case where the flow encoding having the positive polarity is used, and Expression (2) is a case where the flow encoding having the negative polarity is used.

φf(+)=γ*(+)S*Ti*V (1)

φf(−)=γ*(−)S*Ti*V (2)

In the expressions, γ is the gyromagnetic ratio, and S is the applying amount of one gradient magnetic field between the pair of gradient magnetic fields configuring the flow encoding pulse. Ti is the time interval between the centers of each of the pair of gradient magnetic fields configuring the flow encoding pulse. In a case where the gradient magnetic fields are continuously applied, the applying time becomes the same value as that of one gradient magnetic field. Since the transverse magnetization of stationary tissue is V=0, the stationary tissue does not depend on the flow encoding amount and does not receive the phase shift.

In a complex differential image of an image acquired by applying the flow encoding pulse having the positive polarity to a desired axis and an image acquired by applying the flow encoding pulse having the negative polarity to the same axis, a signal from the stationary tissue is deleted due to the differential, and only the signal from blood remains. Thus, a vascular image can be obtained.

From the viewpoint of phase unwrap, when the difference between φf (+) and φf (−) of Expressions (1) and (2) is 180°, that is, in a case of φf=±π/2, the absolute value of the complex differential becomes the maximum. Therefore, when the mean flow velocity V of a blood vessel of an imaging target is designated, if the flow encoding amount (Gvenc) is set to the value determined through the following Expression (3), the signal strength of the blood vessel is imaged with the maximum value.

Gvenc=(γ*S*Ti)=π/(2V) (3)

In Expression (3), in a case where the blood flow velocity V is small, Gvenc may be increased by increasing S or Ti. In a case where the blood flow velocity V is significant, Gvenc may be decreased by decreasing S or Ti. In a general PC method, the flow encoding amount Gvenc is set by using the mean blood flow velocity of a blood vessel which is the imaging target.

FIG. 4 illustrates an example of the cine-imaging sequence (cine-PC sequence) using the pulse sequence of the PC method described above. FIG. 4 illustrates a case of prospective imaging being synchronized with an R-wave of an electro-cardiogram and obtaining images as many as n cardiac time phases as per the elapsed time from the R-wave.

The number of time phases, that is, the number of divisions of the cardiac cycle is, for example, 20. However, the number is not limited. Assuming that the cardiac cycle is one second (1,000 ms), the period of one cardiac time phase becomes 1,000/20=50 ms. Then, an elapsed time of a range from 0 to 50 ms from the R-wave is defined as a first cardiac time phase, and the same of a range from 51 to 100 ms is defined as a second cardiac time phase. In each cardiac time phase, the pulse sequence of the PC method illustrated in FIG. 3 is executed as many as a predetermined number of times.

When a repetition time TR of the pulse sequence in FIG. 3 ranges from 6 to 8 ms, the pulse sequence can be repeated 6 to 8 times in one cardiac time phase. In a case where the axis of the flow encoding has the measurement using the pulse having the positive polarity and the measurement using the pulse having the negative polarity as one set in one axis, data as much as three phase encodings can be collected in one cardiac time phase. If the number of phase encodings in an image is 64, one image can be obtained after approximately 22 seconds. By quantitatively analyzing this cine-image, it is possible to obtain diagnostically important measurements such as the amount of a blood flow passing through the set ROI, and a force of blood striking a vascular wall, that is, wall shear stress.

Here, in a general PC method, in consideration of the mean velocity of the blood flow traveling in the target region, the applying amount (flow encoding amount) of the flow encoding pulse using the pulse sequence of the PC method is set to a uniform value at which the blood flow under the velocity is imaged with high luminance. In other words, in the MRI apparatus, a dynamic range of an image is determined in accordance with the set flow encoding amount. However, as described above, in the cine-PC sequence in which the cardiac cycle is divided and the image for each time phase is obtained, the blood flow velocity varies for each image of the time phase. Therefore, in the uniform flow encoding amount, the ability of imaging a blood vessel is deteriorated depending on the time phase.

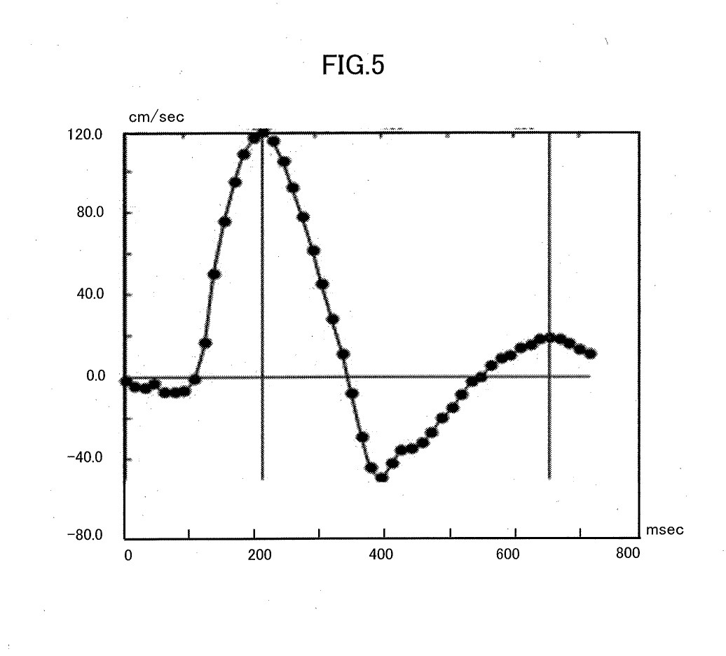

FIG. 5 illustrates an example of a change of the blood flow velocity within one cardiac cycle obtained in the cine-PC sequence. In the diagram, the transverse axis indicates the elapsed time from the R-wave, and the vertical axis indicates the blood flow velocity. As illustrated, the blood flow velocity significantly fluctuates. In a case where the flow encoding amount is set based on the mean blood flow velocity, the ability of imaging a blood vessel is drastically deteriorated. For example, in the time phase in which the blood flow velocity is slow, the signal value is low. In addition, in the time phase in which the blood flow velocity is drastically fast with respect to the set flow encoding amount, due to folding of the phase, the signal value is low similar to when the blood flow velocity is slow. As a result thereof, the reliability of the measurements obtained by quantitatively analyzing the blood flow velocity is also deteriorated.

In the present embodiment, in consideration of the change of the blood flow velocity within the cardiac cycle, the flow encoding amount is caused to be different in at least two time phases and to vary, and the cine-PC sequence is executed. Thus, the ability of imaging a blood vessel in the cine-image is improved. Therefore, in the MRI apparatus of the present embodiment, the control unit has a pre-scanning sequence which is different from the imaging sequence, and in which multiple echo signals are acquired in the time phases different from each other. The pulse computation section calculates the target velocity information from the data for each time phase obtained by performing Fourier transformation for each of the multiple echo signals acquired for each time phase by executing the pre-scanning sequence.

There can be various forms of pre-scanning as long as information indicating the change of the blood flow velocity within the cardiac cycle of the cine-PC sequence can be obtained. Hereinafter, each of embodiments of which the forms of the pre-scanning are different from each other will be described.

### First Embodiment

The MRI apparatus of the present embodiment is characterized in that as the pre-scanning sequence, a pulse sequence which is the same type as the imaging sequence except that the phase encoding is not included, or a pulse sequence which is the same type as the imaging sequence including only low-phase encoding is used.

The flow of the operation of the MRI apparatus of the present embodiment includes the pre-scanning, determination of the flow encoding amount using the pre-scanning data, execution of the cine-PC sequence which is the main imaging, and the image reconstruction. The flow may also include a quantitative analysis of an image obtained in the cine-PC sequence.

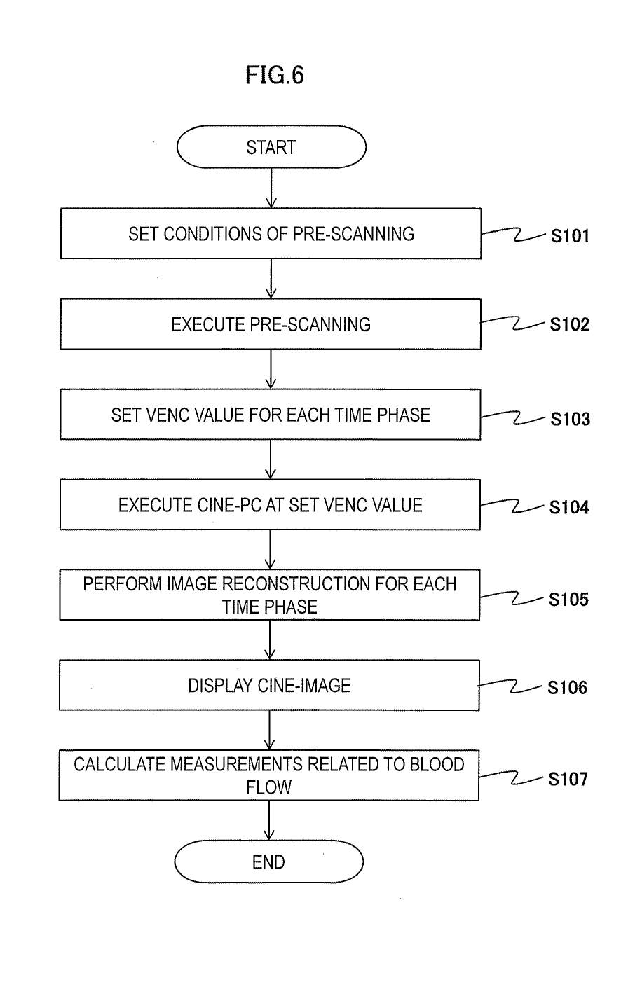

Hereinafter, an operation of the MRI apparatus of the present embodiment will be described with reference to the flow illustrated in FIG. 6.

<<Step S101>>

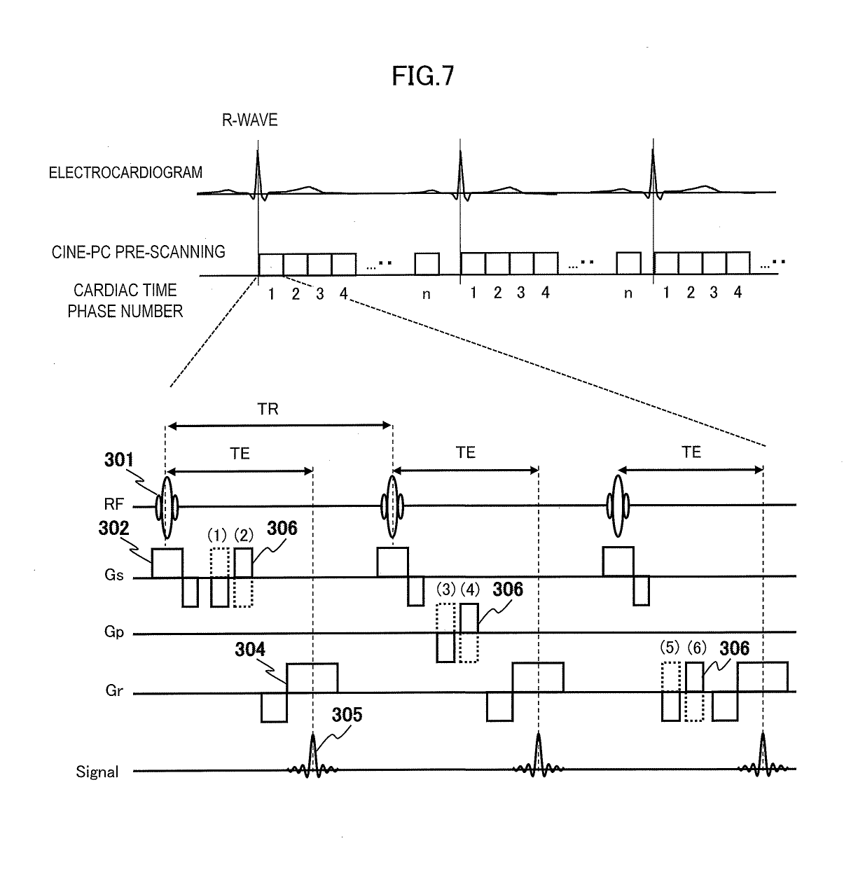

First, the sequence control section 1112 sets the imaging conditions of the pre-scanning. FIG. 7 illustrates an example of the pre-scanning sequence.

The pre-scanning sequence illustrated in FIG. 7 is a sequence of the PC method including applying of the flow encoding gradient magnetic field, similar to the cine-PC sequence illustrated in FIG. 3. However, the phase encoding is not included. In addition, here, it is preferable that the applying axis (applying direction) of the flow encoding gradient magnetic field 306 is the same direction as that of the flow encoding gradient magnetic field of the cine-PC sequence. However, the applying axis is not necessarily the same. FIG. 7 illustrates a case where the flow encoding gradient magnetic field is applied to the axes in three directions of the slice direction Gs, the phase encoding direction Gp, and the lead-out direction Gr. The axis of the flow encoding gradient magnetic field may be in one direction or two directions.

In Step S101, as the imaging conditions of the pre-scanning, in addition to the parameters such as the space resolving power (the number of samplings of the lead-out direction), TE, and TR, the direction of the flow encoding, the number of cardiac time phases, and the flow encoding amount are set.

The space resolving power, TE, TR, and the number of cardiac time phases are set so as to be the same as those of the cine-PC sequence which is the main imaging to be executed thereafter. In addition, the target region of imaging is also the same. As the flow encoding amount, a uniform value, for example, a value optimal for the blood flow velocity of a blood vessel which is the target of the cine-PC sequence (the mean blood flow velocity, the blood flow velocity of the diastolic stage, or the like) is set. In other words, in a case where the pre-scanning is not performed, typical conditions which are registered in the memory in advance as the flow encoding amount of a general cine-PC sequence is read, and the result is set as the flow encoding amount of the pre-scanning.

FIG. 7 illustrates the pre-scanning sequence including no phase encoding. However, the pre-scanning sequence may include low-pass phase encoding. In this case, the phase encoding may be in one direction or two directions. In such cases, 2D data or 3D data can be obtained.

<<Step S102>>

The sequence control section 1112 executes the pre-scanning under the set imaging conditions. The pre-scanning is executed so as to be synchronized with the electro-cardiogram in a state where the object is holding the breath. In FIG. 7, the pre-scanning sequence is illustrated on the lower side, a relationship with respect to the cardiac time phase is indicated with the dotted line. In the pre-scanning sequence illustrated in FIG. 7, since the flow encoding gradient magnetic fields 206 having the positive polarity and the negative polarity are applied in each of three flow encoding directions, applying is required to be repeated 6 times (3*2), and the six repetitive measurements are acquired in one cardiac time phase. For example, when the imaging conditions of the cine-PC include the cardiac cycle to be 960 ms and the number of cardiac time phases to be 16, the time per cardiac time phase becomes 60 ms. In order to acquire the six repetitive measurements in one cardiac time phase, the time per one measurement becomes approximately 10 ms.

Recently, in the cine-PC, since TR ranges from 6 to 8 ms, the pre-scanning described above can be realized in one cardiac time phase.

In a case where the pre-scanning is a sequence of acquiring low frequency region data, for example, when the breath can be held for 10 seconds, 2D pre-scanning data as many as 10 pieces of data can be acquired in the phase encoding direction. In addition, when the breath can be held for 20 seconds, 3D pre-scanning data as many as four pieces of data in the phase encoding direction and as many as four pieces of data in the slice encoding direction can be sufficiently acquired.

The data acquired in the pre-scanning is stored in the memory or a storage device and is used in the next step such that the pulse computation section 1082 calculates the flow encoding amount of the cine-PC sequence.

<<Step S103>>

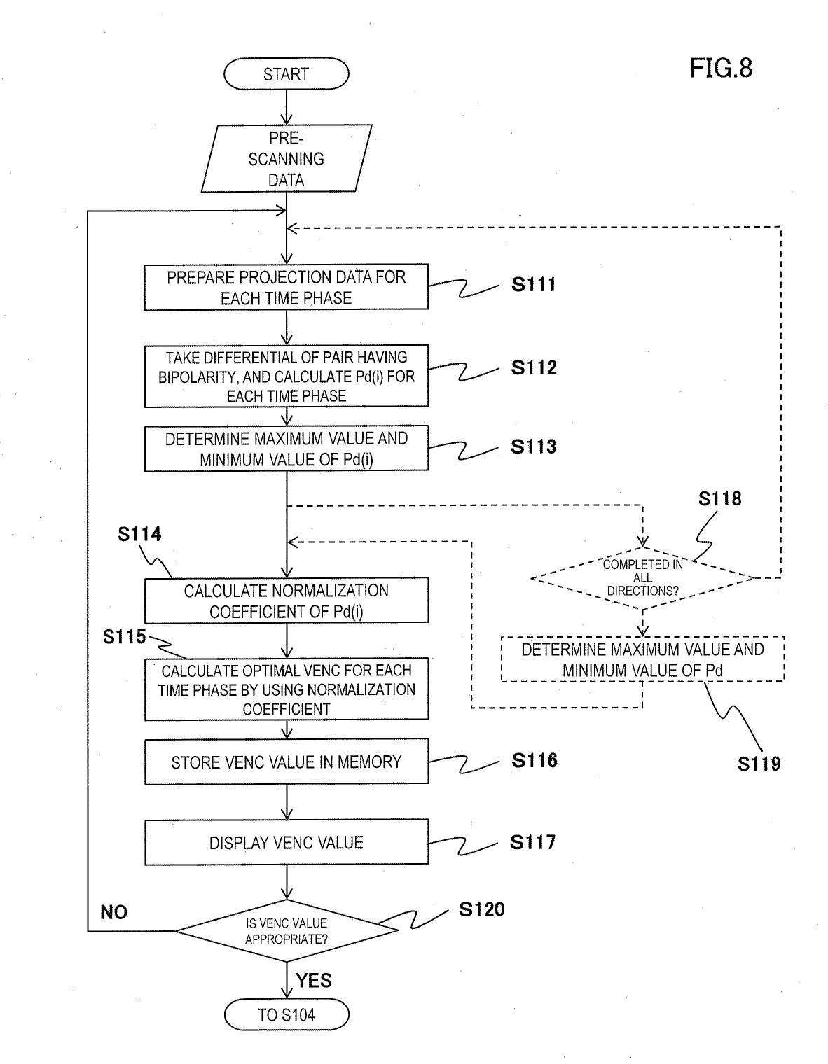

The pulse computation section 1082 calculates a flow encoding amount optimal for each cardiac time phase in the cine-PC sequence based on the pre-scanning data. FIG. 8 illustrates Step S103 in detail. The pre-scanning data acquired in Step S102 is data obtained in the flow encoding direction for each cardiac time phase with respect to each of the flow encoding pulse having the positive polarity and the flow encoding pulse having the negative polarity (both will be collectively referred to as a flow encoding pulse having bipolarity), and the number of pieces of data is 80 (=2*3*16) in the case described above.

First, projection data of the pre-scanning data is prepared (S111). Subsequently, while paying attention to the phase of the projection data, the differential between the pieces of the projection data acquired as the pair of the flow encoding pulse having bipolarity is taken (S112). Hereinafter, data which has taken the differential is caused to be projection of the pre-scanning. In the description below, the data will be expressed as P pro-data Pd(i) (however, d is the flow encoding direction which is any one of Gs, Gp, and Gr (here, for convenience, any one of the x-direction, the y-direction, and the z-direction), and i is 1-n in the cardiac time phase).

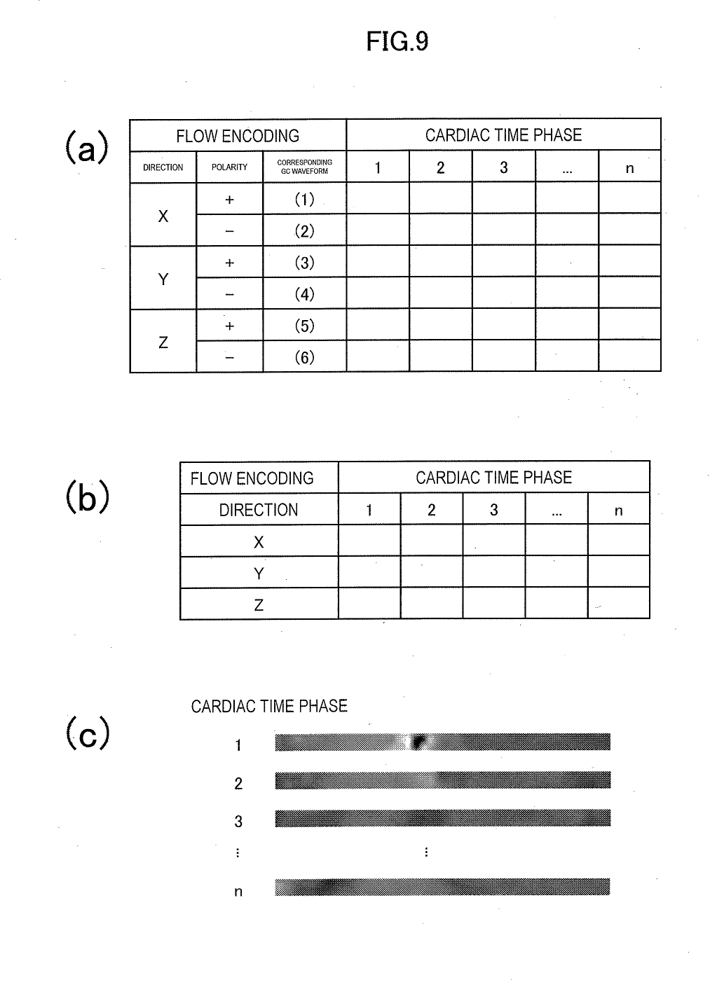

FIG. 9 illustrates a relationship between the pre-scanning data and the projection data. FIG. 9(a) illustrates a table in which the echo signal and the projection data acquired in the pre-scanning are sorted, and FIG. 9 (b) illustrates a table in which the P pro-data Pd(i) is sorted. In a case where Pd(i) is prepared under the conditions equal to those of the cine-PC, the number of Pd(i) is equal to the product of the number of cardiac time phases of the cine-PC and the directions of the flow encoding. In other words, in a case where the flow encoding in the three orthogonal directions with the number of cardiac time phases of 20 is applied, the number of Pd(i) becomes 60.

FIG. 9(c) illustrates an example of the P pro-data Pd(i) in one direction (x-direction). The P pro-data Pd(i) is a phase difference image and the signal strength thereof is equal to the phase difference. In Pd(i) of each time phase, a blood vessel to be the target becomes a high signal when the set flow encoding amount is appropriate. In FIG. 9(c), the high signal can be checked through the image of a cardiac time phase 1. However, in the following cardiac time phase numbers, the signal strength gradually decreases.

Here, regarding the flow encoding amount, by using a relationship of being inversely proportional to the velocity (Expression (3)), the flow encoding amount is optimized so as to be the equally high signal in each cardiac time phase. Therefore, first, a maximum value Max_Pd(i) of the P pro-data Pd (i) is obtained (S113), and by using the value, each Pd(i) is normalized through the following Expression (4) (S114).

St_Pd(i)=Max_Pd(i)/Pd(i) (4)

The value of “St_Pd (i)” obtained as described above is called a normalization coefficient. By using the normalization coefficient, an optimal flow encoding amount (Gvenc) in each time phase is calculated through the following Expression (5) (S115).

Gvenc(i)=Gvenc(0)*St_Pd(i) (5)

Here, Gvenc(0) is the flow encoding amount set in the pre-scanning sequence.

The calculated flow encoding amount is stored in the memory in order to be used as the flow encoding amount of each time phase in the cine-PC sequence which is succeedingly executed (S116).

In a case where the flow encoding of multiple axes is used, the normalization coefficient of each time phase is calculated regarding each of the axes and is stored in the memory. The data area size for retaining the flow encoding amount is one or three in the method in the related art. However, in the present embodiment, the size corresponds to “three directions*the number of cardiac time phases”.

In a case where the flow encoding of multiple axes is used, instead of independently obtaining the normalization coefficient for each axis, a common normalization coefficient can be used. In this case, as indicated with the dotted line in FIG. 8, a maximum value Max_P which is the greatest value among maximum values Max_Px(i), Max_Py(i), and Max_Pz(i) of the axes is obtained (S118, S119), and the normalization coefficient “St_Pd(i)” is calculated through Expression (6).

St_Pd(i)=Max_P/Pd(i) (6)

Calculating an optimal flow encoding amount for each time phase by using the normalization coefficient is similar to the case of independently obtaining the normalization coefficient for each axis.

In Step S113, when the maximum value Max_Pd(i) is obtained, it is preferable that a minimum value Min Pd(i) of the P pro-data Pd(i), the elapsed time (DT: delay time) from the R-wave of the electro-cardiogram which becomes the maximum value or the minimum value, and the like are calculated. The maximum value, the minimum value, and the delay time are stored in the memory 202 (FIG. 2) together with the normalization coefficient calculated in Step S114 (S116). These numerical values can be utilized as indexes of the blood flow velocity when the cine-image is displayed.

The blood flow velocity calculated based on the flow encoding amount of the cardiac time phase taking the maximum value can be considered as the blood flow velocity of the cardiac time phase. Therefore, based on the blood flow velocity, by using the normalization coefficient, the blood flow velocity of each cardiac time phase, and the maximum value or the minimum value of the blood flow velocity may be calculated.

The display unit 113 displays the maximum value and the minimum value of Pd(i) in each flow encoding direction (or the maximum value and the minimum value of the blood flow velocity), the numbers of the cardiac time phases which becomes the maximum value and the minimum value, and the elapsed time from the R-wave which are calculated as described (S117). Accordingly, the operator can check the displayed numerical values, and in a case where the values are determined to be incorrect, the pre-scanning can be executed again (S120).

Hereinabove, Step S103 in FIG. 6 has been described in detail.

<<Step S104>>

Returning to FIG. 6, the sequence control section 1112 starts the cine-PC sequence illustrated in FIG. 4. The cine-PC sequence is repeated until the echo signal having a predetermined number of phase encoding is collected regarding each time phase as well. The echo signal measured by executing the cine-PC sequence is stored in the memory 202 of the CPU 201. In the memory 202, the echo signals are sorted as elements in an array having the cardiac time phase numbers and the flow encoding directions as the dimension. For example, in a case where the cine-PC imaging is executed under the conditions of the number of cardiac time phases of 20 and three directions of the flow encoding, the echo signals are sorted as per the imaging conditions when being acquired. In Step S104, the same sequence as the PC sequence may be executed as a reference sequence except that the flow encoding is not used. In such a case, the sequence includes elements of data array having the number of cardiac time phases of 20 and seven types of flow encoding (three directions of flow encoding*two patterns having bipolarity+no flow encoding).

<<Step S105>>

The image computation section 1081 performs the image reconstruction processing such as Fourier transformation with respect to each of the elements in the data array retained in Step S104, thereby generating image data. Among the pieces of the image data, the phase differential is derived from the pair of the image data having the same flow encoding direction and the different polarity (pair of bipolarity), and the phase differential is retained as a PD image data PCd(i). A PD image is a phase image, and the absolute value image may be prepared at the same time. As the number of pieces of data of The PD image data, there are 60 pieces of the image data under the conditions of the number of cardiac time phases of 20 and three directions of the flow encoding. In addition, when the PD image data PCd(i) is retained, the PD image data PCd(i) is retained by performing mapping with respect to the normalization coefficient St_Pd(i) derived in Step S103 (S114). For example, it is preferable that the normalization coefficient is retained as header information of the image data. The image data generated by using the echo signal which has no flow encoding and is obtained in the reference sequence is a general MR image. The image data is retained as reference image data, without applying the processing described above.

<<S106>>

The display unit 113 displays the image data generated in Step S105 as the cine-image based on controlling of the display control section 1113. In the image of each cardiac time phase in the cine-image, the dynamic range is effectively used in all of the cardiac time phases, and the signal strength of a blood vessel is maximized. In other words, even if the blood flow velocity varies for each cardiac time phase, the image of each cardiac time phase is imaged as a high signal at all times.

Meanwhile, even though the signal strength of all of the time phases is maximized, the blood flow velocity cannot be visually grasped from the luminance value (signal strength) of the image, and the measurements related to the blood flow velocity and the blood flow movement cannot be directly derived from the signal strength. Therefore, in the present embodiment, the index of the blood flow velocity is displayed together with the cine-image. As the index of the blood flow velocity, the normalization coefficient calculated in S115 can be used.

The meaning of displaying the normalization coefficient as the index of the blood flow velocity will be described.

In a case where the cine-PC imaging is performed with the uniform flow encoding amount, the signal strength varies in proportion to the blood flow velocity. The variation leads to the deterioration of the ability of imaging a blood flow. Meanwhile, by utilizing the characteristics of the blood flow velocity being proportional to the signal strength, the images of the high signal are visually checked among a series of displayed cine-PC images, and the cardiac time phase having the fast blood flow velocity can be specified. In the MRI apparatus of the present embodiment, since the flow encoding amount is changed such that the signal strength becomes the high signal in each cardiac time phase, the cardiac time phase having the fast blood flow velocity cannot be visually checked. The normalization coefficient is a coefficient for causing the signal strength (Pd (i)) varying for each time phase in proportion to the blood flow velocity to be aligned in a uniform value. The normalization coefficient is proportional to the inverse number of the velocity. Therefore, the normalization coefficient is retained as the header information of the image and is displayed, and thus, a user can be provided with information related to the variation of the velocity for each cardiac time phase which cannot be discriminated from the signal strength.

As a specific example, an example of a cardiac time phase 1 having the blood flow velocity of 100 cm/second and a cardiac time phase 2 having the blood flow velocity of 25 cm/second will be described. The signal strength of the cine-PC image (image of a target blood vessel, the same hereinafter) is a phase value, and the dynamic range thereof is generally ±180 degrees. Therefore, in a case of the uniform flow encoding amount (method in the related art), when the signal strength of the cine-PC image of the cardiac time phase 1 (blood flow velocity of 100 cm/second) is set to 180, the signal strength of the cine-PC image of the cardiac time phase 2 (blood flow velocity of 25 cm/second) becomes 45. In the method in the related art, there is no concept of the normalization coefficient. However, when the normalization coefficient is applied to the cine-PC image, both the cardiac time phase 1 and the cardiac time phase 2 becomes “1”.

Meanwhile, in the present embodiment, the flow encoding amount is changed for each cardiac time phase, and the signal strength of the cine-PC image of both the cardiac time phase 1 and the cardiac time phase 2 is set to 180. That is, in the cardiac time phase 1 (blood flow velocity of 100 cm/second), the cine-PC image has the signal strength of 180 and the normalization coefficient of 1. In the cardiac time phase 2 (blood flow velocity of 25 cm/second), the cine-PC image has the signal strength of 180 and the normalization coefficient of 4. In this manner, in the present embodiment, by effectively utilizing the dynamic range, a blood flow can be imaged with high luminance in the cine-PC image in all of the time phases, and the blood flow velocity of each time phase can be grasped through the normalization coefficient.

As the index of the blood flow velocity, instead of the normalization coefficient or in addition to the normalization coefficient, the inverse number of the normalization coefficient, the set flow encoding amount in the cine-PC sequence for each time phase, and the like can be held as the header information of the image data or can also be displayed.

<<Step S107>>

As necessary, the cine-PC image data is analyzed, and the measurement related to a blood flow is calculated. For example, the time integration of the blood flow velocity V (cm/s) can be obtained from the blood flow velocity for each time phase obtained from the cine-PC image data (graph illustrated in FIG. 5), and by using a cross-sectional area A (cm²) of a blood vessel, an amount Q of a blood flow (cm³) can be calculated through Expression (7).

Q=A*∫vdt (7)

The cross-sectional area of a blood vessel can be obtained as an area of the ROI.

In addition, a force of blood striking a vascular wall is called the wall shear stress and is obtained as the product of the viscosity coefficient of the fluid and the velocity gradient of the wall surface.

In this manner, by utilizing the image data of the cine-PC, hemodynamic movement can be quantitatively analyzed.

As described above, according to the MRI apparatus of the present embodiment, by performing the pre-scanning, the flow encoding amount applied to each time phase in the cine-PC imaging which is the main imaging is calculated, and the flow encoding amount is caused to be different in at least two time phases. Thus, imaging can be performed by using a flow encoding amount optimal for the blood flow velocity at the moment for each time phase in the cine-PC imaging. Accordingly, the signal value of the target blood vessel is decreased depending on the time phase, and the problem of deterioration of the accuracy of the obtained blood flow velocity can be solved. In addition, the blood vessel can be imaged with high signal strength throughout the entire cardiac cycle.

In addition, according to the present embodiment, when the cine-PC image data is stored in the memory or the storage device, the normalization coefficient or the flow encoding amount which becomes the index of the blood flow velocity is imparted as supplementary information of the cine-PC image for each time phase. Therefore, intuitive grasping of the blood flow velocity through a change of the signal value in the cine-image can be compensated for.

### Second Embodiment

An MRI apparatus of the present embodiment is the same as the first embodiment for executing the pre-scanning sequence similar to the cine-PC sequence. The present embodiment is different therefrom in that the number of time phases in the pre-scanning sequence and the number of time phases in the cine-PC sequence are different from each other.

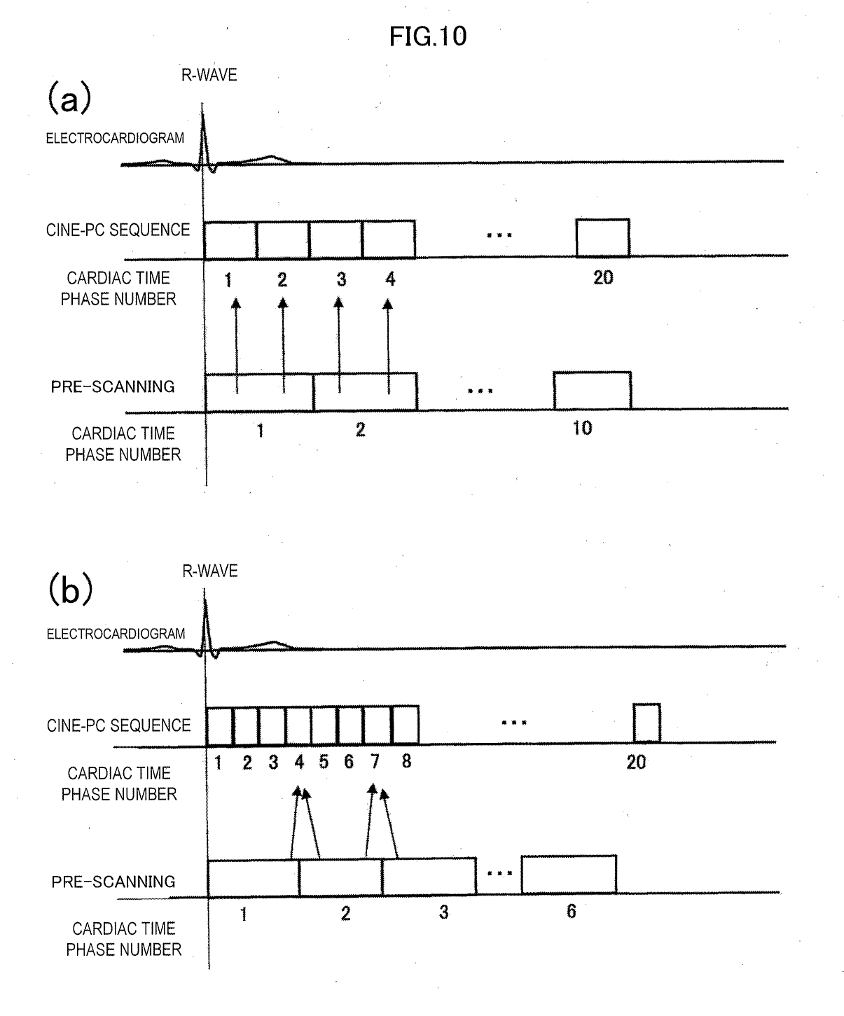

The cine-PC sequence and the pre-scanning sequence are electrocardiographic synchronous prospective imaging sequences respectively illustrated in FIGS. 4 and 7. However, the number of time phases in the pre-scanning sequence is smaller than the number of time phases in the cine-PC sequence. FIG. 10 illustrates a relationship between the time phase in the cine-PC sequence and the time phase in the pre-scanning sequence. The illustrated example shows a case (FIG. 10(a)) where the number of time phases in the pre-scanning sequence is 10 and the number of time phases in the cine-PC sequence is 20, and a case (FIG. 10(b)) where the number of time phases in the pre-scanning sequence is 6 and the number of time phases in the cine-PC sequence is 20.

In the present embodiment as well, since calculating the flow encoding amount of each cardiac time phase in the cine-PC sequence by using the pre-scanning data acquired through the pre-scanning is similar to that of the first embodiment, description will be given by quoting the flow in FIG. 8. As illustrated in FIG. 8, first, the projection data of the pre-scanning is prepared (S111), and the differential between the pair of flow encoding having bipolarity of which the flow encoding direction is the same is taken from the projection data, thereby calculating P pro-data Pd(j) (j ranges from 1 to min the cardiac time phase in the pre-scanning) (S112).

Subsequently, the maximum value and the minimum value of Pd(j) are determined (S113), and the normalization coefficient for each cardiac time phase is calculated by using the maximum value (S114). In this case, in a case where the flow encoding has multiple directions, the maximum value and the minimum value are obtained from the maximum values and the minimum values in all of the directions, and the normalization coefficient is calculated. The flow encoding amount of each cardiac time phase in the cine-PC sequence is calculated by using the normalization coefficient (S115). In this case, the number of pieces of data of the normalization coefficient is the same as the number m of cardiac time phases in the pre-scanning and is smaller than the number of pieces of data of the flow encoding amount to be calculated (same as the number n of cardiac time phases in the cine-PC sequence). Therefore, after mapping of the cardiac time phases of both thereof is performed, the flow encoding amount is calculated.

Various types of method can be considered for the mapping. As a method, for example, the time phases (multiple) of the cine-PC included within the time of the time phase (j) in the pre-scanning uses the normalization coefficient of the time phase (j) of the pre-scanning. As illustrated in FIG. 10(a), in a case where the number of time phases of the cine-PC is the integral multiplication of the number of time phases in the pre-scanning, mapping of all of the time phases is performed through this method. In addition, as illustrated in FIG. 10(b), in a case where the time phase (i) of the cine-PC straddles two time phases (j), that is, the time phases (j+1) and (j−1) in the pre-scanning, the mean value of the normalization coefficients of the two time phases is used.

In the example illustrated in FIG. 10(b), the cardiac time phase 4 of the cine-PC uses the mean value of the cardiac time phase 1 and the cardiac time phase 2 in the pre-scanning, and the cardiac time phase 7 of the cine-PC uses the mean value of the cardiac time phase 2 and a cardiac time phase 3 in the pre-scanning. The mean may be a simple mean or may be a weighted mean obtained in accordance with the overlapping degree between the time phase in the pre-scanning and the two time phases of the cine-PC. For example, in the weighting, the time difference between the time centers of two cardiac time phases adjacent to each other in the pre-scanning with respect to the time centers of the cardiac time phases in the cine-PC sequence is derived, and the weighting is performed in accordance with the ratio of the time difference.

As described above, after the flow encoding amount is calculated by using the normalization coefficient, the result is stored in the memory (S116), thereby being used as the flow encoding amount of each cardiac time phase of the cine-PC which is succeedingly executed. Thereafter, execution of the cine-PC at the flow encoding amount set for each cardiac time phase and the image reconstruction are similar to those of the first embodiment.

In the present embodiment, for example, as illustrated in FIG. 10(b), when the cardiac cycle is divided into six sections in total such as prophase-metaphase-anaphase of a systolic stage and prophase-metaphase-anaphase of a diastolic stage, the number of cardiac time phases in the pre-scanning can be drastically reduced compared to the number of cardiac time phases in the cine-PC imaging. In this case as well, mapping of the cardiac time phases in the cine-PC imaging and the cardiac time phases in the pre-scanning can be performed through the technique described above. This embodiment is useful for an imaging target having a small change of the blood flow velocity.

According to the present embodiment, when the number of divisions of the cardiac cycle in the pre-scanning is reduced, the interval of one cardiac time phase is elongated. Therefore, the degree of freedom of setting the parameter of the pre-scanning sequence is high. In addition, as described in the first embodiment, the pre-scanning can be employed not only in a sequence in which the phase encoding is not used but also in a sequence in which the low-pass phase encoding is used. However, in the present embodiment, since the interval of the cardiac time phase can be elongated, low-pass pre-scanning data can be acquired without extending the measurement time for the pre-scanning.

### Third Embodiment

An MRI apparatus of the present embodiment uses a sequence of type different from that of the cine-PC sequence, as the pre-scanning sequence. Specifically, a sequence of a two-dimensional space selection excitation method is employed. The two-dimensional space selection excitation method is an imaging method different from the excitation of a slice surface performed by the combination of a slice selection gradient magnetic field and the RF pulse. In the two-dimensional space selection excitation method, a vibration gradient magnetic field of two directions and the RF pulse (here, will be referred to as a two-dimensional selection RF pulse) are combined together, an arbitrary region having a cylindrical shape is selectively excited, and image forming is performed by obtaining an echo signal from the region thereof.

As an example in which the two-dimensional space selection excitation method is applied to vascular imaging, for example, NPL 1 discloses an example in which the two-dimensional space selection excitation method is used for the purpose of restraining a signal. However, in the present embodiment, the two-dimensional excitation method is utilized for acquiring the pre-scanning data.

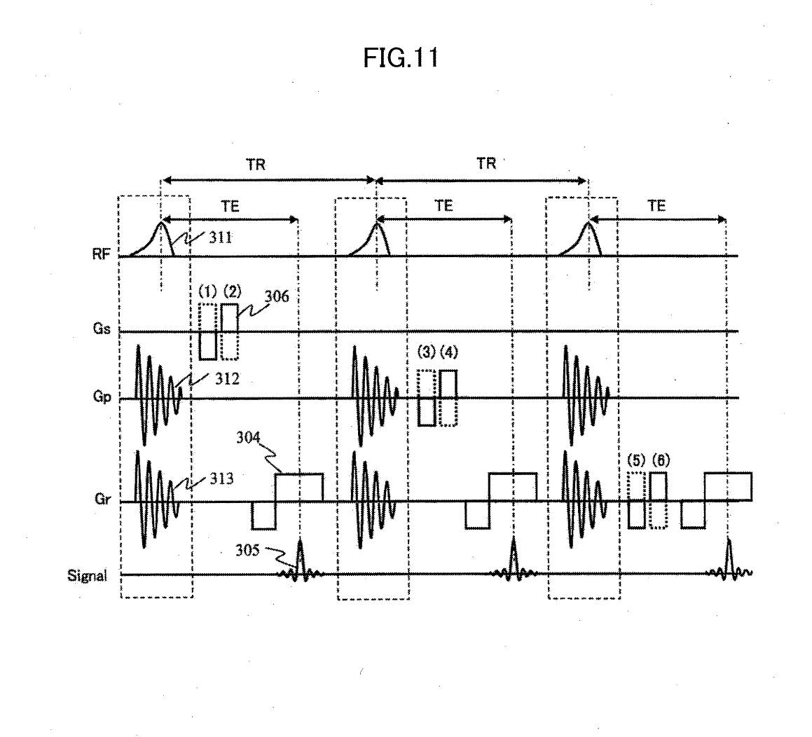

FIG. 11 illustrates an example of a sequence of the two-dimensional selection excitation method. The sequence is the same as the pre-scanning sequence illustrated in FIG. 7, except the place related to two-dimensional excitation surrounded by the square which is indicated with the dotted line. The same elements are indicated with the same reference signs. In the sequence of the two-dimensional excitation method, image forming of a desired region can be selectively performed by appropriately setting the frequency and the strength of an RF pulse 311, and gradient magnetic field waveforms 312 and 313 in a Gp direction and a Gr direction.

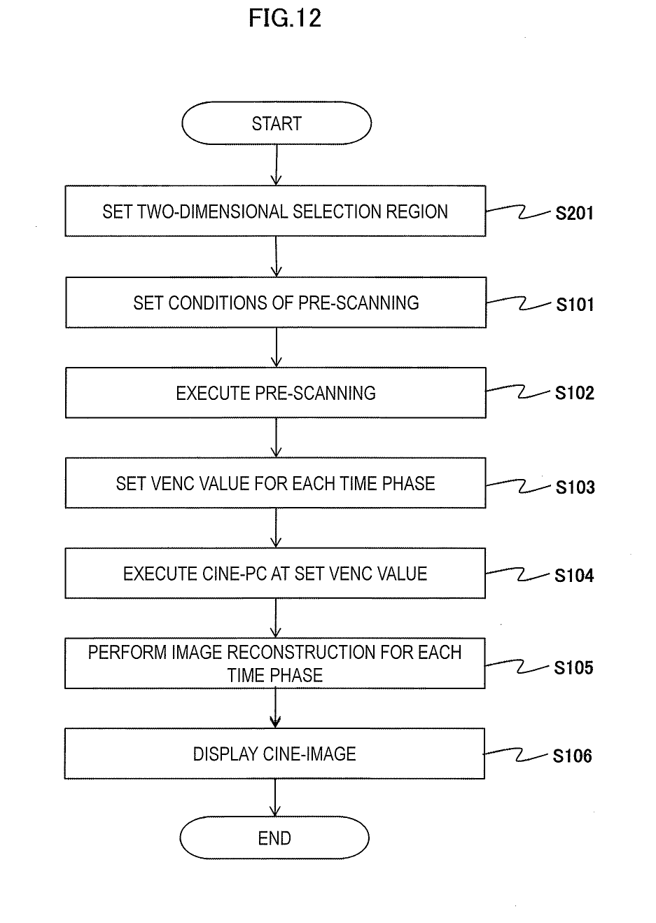

FIG. 12 illustrates a processing procedure in the control unit 111 and the computation unit 108 of the present embodiment. In FIG. 12, the same processing as the processing illustrated in FIGS. 6 and 8 is indicated with the same reference signs, and the detailed description will be omitted.

<<Step S201>>

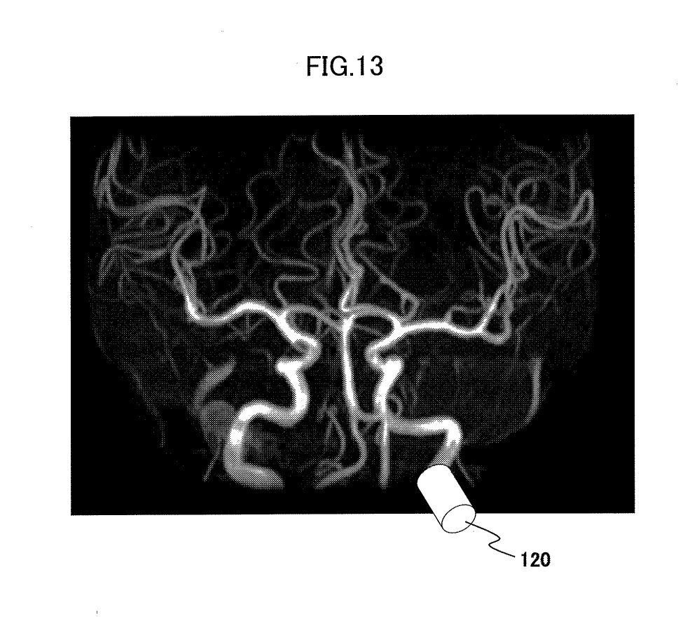

The control unit 111 receives setting of a region performed via a UI by a user. For example, the user checks the blood vessel of interest with reference to an image for positioning and selects the region such that the region becomes orthogonal to the traveling of the blood vessel of interest. Examples of the blood vessel of interest include a bifurcated portion of the blood vessel and arterial cancer. FIG. 13 illustrates an example of the UI in which the blood vessel of interest is selected. In FIG. 13, a cylindrical region 120 is set in the blood vessel slightly to the right at the lower center so as to be orthogonal to the vascular traveling direction. Since the region is orthogonal to traveling of the blood vessel, the two-dimensional excitation pulse used in the pre-scanning intersects the blood flow inside the blood vessel and the volume of the region is reduced, it is possible to expect to more precisely measure the blood flow velocity in the blood vessel of interest.

When the radius and the orientation of the selected region are specified, the sequence of the two-dimensional space selection excitation method which is the pre-scanning sequence is calculated. Specifically, the two-dimensional excitation pulse and the waveform of the gradient magnetic field are calculated. For example, this calculation may be a function of the pulse computation section 1082 and may be a function of the sequence control section 1112.

<<Step S101>>

TE, TR, the number of cardiac time phases, the direction of the flow encoding, and the like in the pre-scanning are set. The number of cardiac time phases may be the same as the number of time phases in the cine-PC sequence which is the main imaging and may be different therefrom. Generally, in the two-dimensional space selection excitation method, since TR is required to be longer than the sequence of the PC method illustrated in FIG. 7, processing such as reducing the number of cardiac time phases so as to correspond thereto and deriving the parameter value at which the extension of TR becomes the minimum is performed.

<<Steps S102 to S106>>

Executing the pre-scanning in which the two-dimensional space selection excitation method is applied under the set conditions, executing the cine-PC imaging by using the acquired pre-scanning data, and combining the normalization coefficient calculated when setting VENC with the cine-image data as the header information at that time are similar to those of the first and second embodiments. However, in Step S103, processing of mapping the result of the blood flow velocity obtained in the pre-scanning with the flow encoding amount of the cine-PC is executed. In this processing, the processing is performed because there is a difference in the number of cardiac time phases, the delay time or the period from the R-wave of each cardiac time phase between the pre-scanning and the cine-PC since TR is different between the pre-scanning and the cine-PC. The processing can be performed through a method similar to the mapping of the time phases in the second embodiment.

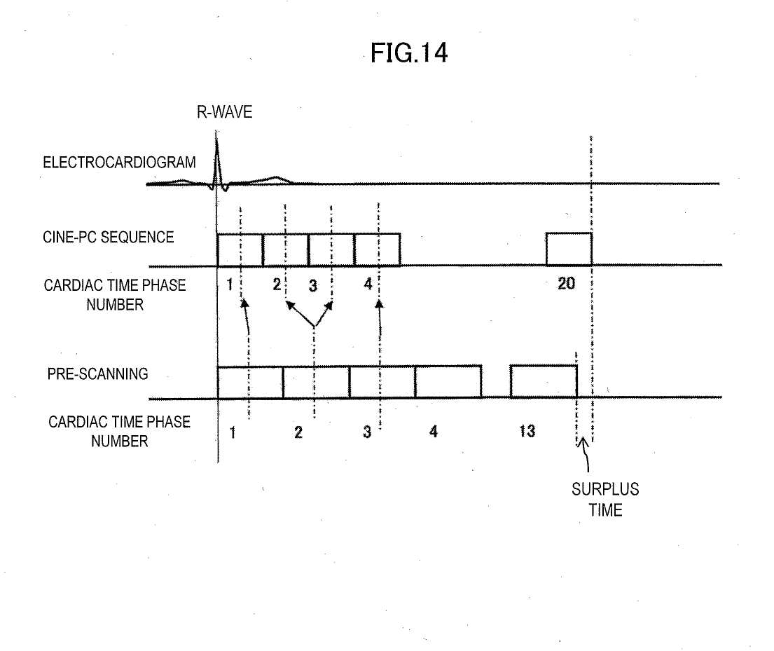

For example, as illustrated in FIG. 14, when the cardiac cycle is one second and the number of cardiac time phases of the cine-PC is 20, the time per cardiac time phase is 50 ms. In the pre-scanning, in a case where the number of cardiac time phases is 13 with respect to the same cardiac cycle, the number of one cardiac time phases becomes 76 ms. Here, 12 ms of the end number (50 ms*20−76 ms*13) is allocated as the surplus time after the 13th cardiac time phase.

In this case, the time center is derived regarding the pre-scanning and each cardiac time phase of the cine-PC. Ina case where the flow encoding amount of the cardiac time phase (i) of the cine-PC is determined, the cardiac time phase (j) in the pre-scanning having the time center of which the time difference becomes the smallest with respect to the time center of the cardiac time phase (i) of the cine-PC is determined. Subsequently, with reference to the blood flow velocity of the cardiac time phase (j) in the pre-scanning, the flow encoding amount to be converted is set as the imaging conditions when the cardiac time phase (i) of the cine-PC is acquired.

The processing is inserted between S114 and S115 in the flow of FIG. 8 illustrating Step S103 in detail.

According to the present embodiment, when the two-dimensional space selection excitation method in which the high frequency magnetic field can be applied to the cylindrical region is applied to the pre-scanning, the pre-scanning data can be collected from only the blood vessel of interest. Accordingly, the blood flow velocity in the blood vessel of interest can be more precisely measured, and an optimal flow encoding amount can be applied to the imaging conditions of the cine-PC. The present embodiment is particularly suitable for a bifurcated portion of a blood vessel or arterial cancer in which it is important to obtain the blood flow velocity of the blood vessel with high accuracy.

### Fourth Embodiment

In the first to third embodiments described above, descriptions are mainly given regarding a case of being applied to the prospective imaging method in which the echo signals are allocated to the cardiac time phases set as per the elapsed time from the R-wave. However, in the embodiments, the R-wave set in consideration of the fluctuation of the heart rate and the time interval of the R-wave can be divided into predetermined cardiac time phases, and the embodiments can also be applied to a retrospective imaging method in which the echo signals are allocated.

In the present embodiment as well, first, the pre-scanning is executed, the flow encoding amount of each cardiac time phase of the cine-PC imaging is calculated, and the calculated flow encoding amount is set to the flow encoding amount of each cardiac time phase in the cine-PC imaging. The pre-scanning may be the same as the cine-PC imaging and may be a sequence of the two-dimensional space selection excitation method. In addition, the method of calculating the flow encoding amount is similar to that of the first embodiment. In the retrospective imaging, based on the mean value of the intervals of the cardiac cycle, the cardiac cycle is divided by the number of cardiac time phases set in advance. Therefore, the flow encoding amount calculated from the pre-scanning data is set to the cardiac time phases.

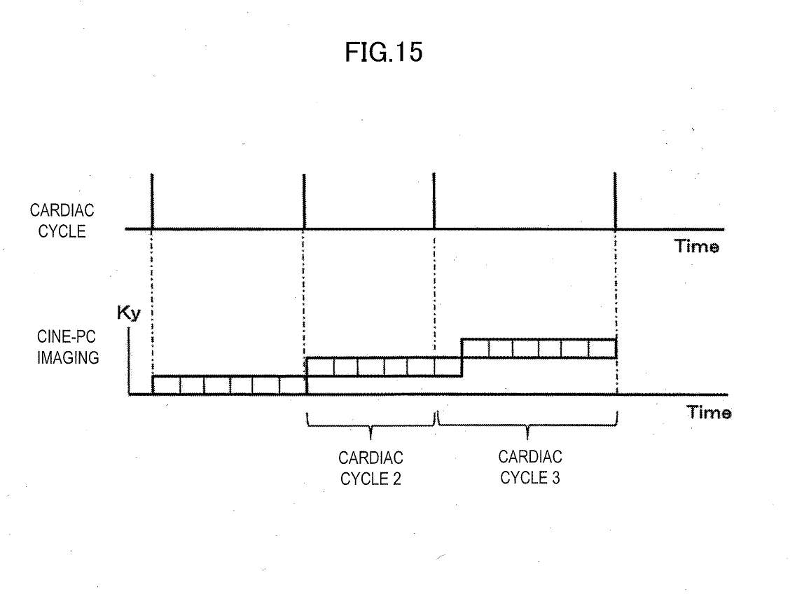

FIG. 15 illustrates an example of the cine-PC imaging performed through the retrospective imaging method. As an example, FIG. 15 illustrates a case of six divisions, three cardiac cycles, and measuring the signals of all of the phase encodings.

In a cardiac cycle 1 having the same interval as the mean value of the cardiac cycle, data as much as six cardiac time phases can be obtained. However, in a cardiac cycle 2 shorter than the mean value, data as much as the cardiac time phases set in advance cannot be obtained. In a cardiac cycle 3 longer than the mean value, data more than the cardiac time phases set in advance can be obtained. In the retrospective imaging, regarding the cardiac cycle shorter than the mean value or the cardiac cycle longer than the mean value as well, the data obtained in the cardiac cycle is divided into the number of cardiac time phases (Here, six) set based on the mean value and is handled as the data of each cardiac time phase. For example, in the cardiac cycle 2, the data as much as five cardiac time phases is divided into six cardiac time phases, and in the cardiac cycle 3, the data as much as seven cardiac time phases is divided into six cardiac time phases, thereby being respectively handled as the data of one to six cardiac time phases. Therefore, a loss and a surplus (overlapping) are generated in the data of each cardiac time phase. However, the loss of the data is covered by repeating the measurement.

In a case where the loss of the data is covered, the phase encoding amount has the precedence. For example, in a case where a loss of the phase encoding amount occurs in a cardiac time phase n, the data is compensated for from the cardiac time phases such as a cardiac time phase n−1 and a cardiac time phase n+1 which are adjacent thereto. In this case, the echo signal having the small time difference between the cardiac time phases is preferentially employed. In a case where there are echo signals of which the time differences between the cardiac time phases are the same as each other, the echo signal having the small difference between the flow encoding amounts is employed. In addition, for example, in a case where the difference between the flow encoding amounts exceeds a threshold value set in advance, a rule of not employing the echo signal of the cardiac time phase thereof may be applied.

In addition, overlapping data may be deleted. However, in this case as well, the data having the small difference between the flow encoding amount and the flow encoding amount set to the cardiac time phase to be compensated for is employed.

As described above, by applying the rule of compensating the loss of the phase encoding amount and deleting the overlapping data, it is possible to obtain data in which the flow encoding amount set for each cardiac time phase does not significantly vary.

As another method of compensating the data, the loss of the echo signal may be estimated by using the signal of a low frequency region (region in which the phase encoding amount is close to zero) satisfying the phase encoding amount and the flow encoding amount, and applying so-called half-Fourier processing.

According to the present embodiment, in the retrospective imaging as well, the signal value of a blood flow depended on the cardiac time phase can be prevented from being deteriorated, and the ability of imaging a blood flow can be improved.

<Execution Form of Display>

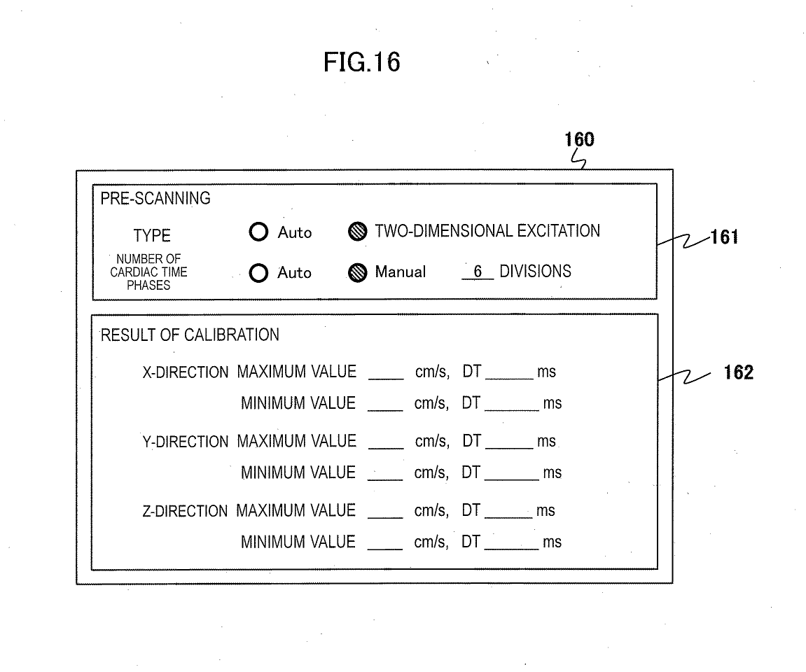

Subsequently, description will be given regarding an execution form of a display unit which displays the UI for inputting the imaging conditions or a computation result of the computation unit when executing each of the embodiments described above. FIG. 16 illustrates an example of a display screen.

A screen 160 is divided into a condition input unit 161 for inputting the conditions of the pre-scanning, and a result display unit 162 for displaying a result of the computation unit. For example, the screen 160 is displayed when the cine-PC imaging is selected as the imaging sequence.

An operator inputs whether the type of the pre-scanning, that is, the same conditions as the cine-PC is applied or the two-dimensional excitation method is applied via the condition input unit 161. The items indicated with black circles in the diagram illustrate items designated by the operator. In FIG. 16, the two-dimensional space selection excitation method is selected. Subsequently, regarding the number of cardiac time phases in the pre-scanning, inputting whether “Auto” is selected and the same imaging conditions as the cine-PC are applied or “Manual” is selected and the values different from those of the cine-PC are applied is performed. In FIG. 16, “Manual” is selected, and “six divisions” is designated as the number of divisions of the cardiac cycle.

For example, when the two-dimensional space selection excitation method is selected, the image illustrated in FIG. 13 is displayed, and thus, the position for two-dimensional excitation can be designated. Thereafter, when the pre-scanning is executed under the set conditions, Step S103 (flow in FIG. 8) illustrated in FIG. 6 is executed, and the value calculated by the pulse computation section 1082 is displayed as a result of calibration. In other words, the maximum value and the minimum value of the blood flow velocity in each of the flow encoding directions, and the delay time (DT) from the R-wave of the electro-cardiogram which becomes the value thereof are automatically Calculated and are displayed within the display screen.

The numerical values are used when the computation unit 108 calculates the measurements related to the blood flow movement, and can also be used as the guidelines for performing the pre-scanning again when being checked by the operator. For example, there may be a case where the accuracy of data obtained through the pre-scanning is deteriorated when the blood vessels overlap each other, thereby leading to an incorrect value. However, since the values are displayed, the pre-scanning can be executed again before the main imaging.

The display screen illustrated in FIG. 16 is an example. Therefore, on the display screen, items other than the illustrated items, the image for determining the excitation position, and the like can also be displayed. Moreover, in the method of displaying the calibration result as well, not only the numerical values but also a graphical display and the like can be employed.

According to the present embodiment, the operations of the MRI apparatus described in the first to fourth embodiments can be customized and executed by the operator.

As described above, according to the MRI apparatus of the present embodiment, the signal of a blood flow depended on the cardiac time phase can be prevented from being deteriorated, the ability of imaging a blood flow can be enhanced in all of the cardiac time phases, and the blood flow velocity can be calculated with high accuracy.

### REFERENCE SIGNS LIST

100 MRI APPARATUS, 101 OBJECT, 102 STATIC MAGNETIC FIELD GENERATING MAGNET, 103 GRADIENT MAGNETIC FIELD COIL, 104 RF COIL, 105 RF PROBE, 106 SIGNAL DETECTION UNIT, 107 SIGNAL PROCESSING UNIT, 108 COMPUTATION UNIT, 109 GRADIENT MAGNETIC FIELD POWER SUPPLY, 110 TRANSMISSION UNIT, 111 CONTROL UNIT, 112 BED, 113 DISPLAY UNIT, 114 INPUT UNIT, 115 MEASUREMENT INSTRUMENT, 201 CPU, 202 MEMORY, 203 STORAGE DEVICE, 1081 IMAGE COMPUTATION SECTION, 1082 PULSE COMPUTATION SECTION, 1083 ROI SETTING SECTION, 1111 MAIN CONTROL SECTION, 1112 SEQUENCE CONTROL SECTION, 1113 DISPLAY CONTROL SECTION.

1. A magnetic resonance imaging apparatus comprising:
a magnetic resonance imaging unit that collects a magnetic resonance signal;
a control unit that controls the magnetic resonance imaging unit as per a pulse sequence; and
a computation unit that prepares an image of a test target by using the magnetic resonance signal collected by the magnetic resonance imaging unit, and time phase information related to a cyclic motion of the test target,
wherein the control unit has an imaging sequence which serves as the pulse sequence, which includes applying of a flow encoding pulse, and in which an echo signal is acquired for each time phase, and
wherein an applying amount of the flow encoding pulse in the imaging sequence is controlled so as to be different in at least two time phases.

2. The magnetic resonance imaging apparatus according to claim 1, further comprising:
an input unit that receives the time phase information,
wherein the control unit controls the imaging sequence by using the time phase information received by the input unit.

3. The magnetic resonance imaging apparatus according to claim 1,
wherein the computation unit causes data acquired in the imaging sequence to be data for each time phase by sorting the data in the order of an elapsed time while having one time point in the time phase information as a starting point.

4. The magnetic resonance imaging apparatus according to claim 1,
wherein the computation unit includes a pulse computation section which calculates the applying amount of the flow encoding pulse for each time phase based on velocity information of a fluid included in the test target for each time phase.

5. The magnetic resonance imaging apparatus according to claim 4,
wherein the control unit has a pre-scanning sequence which is different from the imaging sequence, which includes applying of the flow encoding pulse, and in which the echo signal is acquired for each time phase, and
wherein the pulse computation section calculates the velocity information of the fluid based on projection data of the echo signal acquired for each time phase by executing the pre-scanning sequence.

6. The magnetic resonance imaging apparatus according to claim 5,
wherein the pre-scanning sequence is a pulse sequence which is the same type as the imaging sequence except that phase encoding is not included, or is a pulse sequence which is the same type as the imaging sequence including only low-phase encoding.

7. The magnetic resonance imaging apparatus according to claim 5,
wherein the computation unit includes an ROI setting section which receives setting of ROI regarding the test target, and the pulse computation section calculates the velocity information of the fluid in the ROI set in the ROI setting section.

8. The magnetic resonance imaging apparatus according to claim 5,
wherein the pre-scanning sequence is a sequence which includes excitation caused by a two-dimensional excitation pulse and in which the magnetic resonance signal from a region excited by the two-dimensional excitation pulse is acquired.

9. The magnetic resonance imaging apparatus according to claim 5,
wherein the number of time phases in the pre-scanning sequence and the number of time phases in the imaging sequence are different from each other.

10. The magnetic resonance imaging apparatus according to claim 4,
wherein the pulse computation section includes a normalization coefficient calculating section which calculates a normalization coefficient of the applying amount of the flow encoding pulse calculated for each time phase.

11. The magnetic resonance imaging apparatus according to claim 10, further comprising:
a display unit that displays a processing result of a signal processing unit,
wherein the display unit displays at least one of the applying amount of the flow encoding pulse, the velocity information of the fluid, and the normalization coefficient, together with an image prepared for each time phase.

12. The magnetic resonance imaging apparatus according to claim 1,
wherein the imaging sequence includes a multi-directional flow encoding pulse, and
wherein the control unit controls the applying amount of the flow encoding pulse independently in multiple directions.

13. A blood flow imaging method in which a magnetic resonance image for each time phase is acquired by executing a pulse sequence including a flow encoding pulse with reference to time phase information related to a cyclic motion of a test target,
wherein an applying amount of the flow encoding pulse is caused to be different in at least two time phases.

14. The blood flow imaging method according to claim 13,
wherein the applying amount of the flow encoding pulse is caused to be different in accordance with a blood flow velocity of a blood flow flowing in the test target.

15. The blood flow imaging method according to claim 13,
wherein the time phase is determined as per an elapsed time from an R-wave in an electro-cardiogram.

16. The blood flow imaging method according to claim 13,
wherein the time phase is determined by dividing the R-wave into intervals based on a mean value of the intervals of the R-wave in the electro-cardiogram.
